(19) United States
(12) Patent Application Publication
(10) Pub. No.: US 2016/0256371 A1
(43) Pub. Date: Sep. 8, 2016

(54) PHOTOSTABLE COMPOSITIONS COMPRISING PARA-ALKOXYL PHENYL SUBSTITUTED PROPENOIC ACID (APP) DERIVATIVES

(71) Applicant: HALLSTAR INNOVATIONS CORP., Chicago, IL (US)

(72) Inventors: Hui Feng, Suzhou (CN); Shengkui Hu, Darien, IL (US)

(21) Appl. No.: 15/061,599

(22) Filed: Mar. 4, 2016

Related U.S. Application Data
(60) Provisional application No. 62/128,499, filed on Mar. 4, 2015.

(51) Int. Cl. A61K 8/37; C09D 5/32; C09D 7/12; C09K 15/06; A61Q 17/04; C08K 5/101

(52) U.S. Cl. CPC A61K 8/37; A61Q 17/04; C08K 5/101; C09D 7/1241; C09K 15/06; C09D 5/32

(57) ABSTRACT

The present disclosure relates, according to some embodiments, to photostable UV absorbing compositions comprising para-alkoxyl phenyl substituted propenoic acid (APP) derivatives. Furthermore, the present disclosure relates to methods of prolonging the UV absorption capabilities of a composition using photostable UV absorbing compositions comprising APP derivatives.

### CROSS-REFERENCE TO RELATED APPLICATIONS

This application claims priority to U.S. Provisional Patent Application No. 62/128,499 filed on Mar. 4, 2015. The contents of the above application is hereby incorporated in it's entirety by reference.

### FIELD OF THE DISCLOSURE

The present disclosure relates to photostable UV absorbing compositions comprising para-alkoxyl phenyl substituted propenoic acid (APP) derivatives. Furthermore, the present disclosure relates to methods of prolonging the UV absorption capabilities of a composition using photostable UV absorbing compositions comprising APP derivatives.

### BACKGROUND OF THE DISCLOSURE

Increased public awareness regarding the hazards associated with exposure of human skin to ultraviolet (UV) radiation has contributed to the increasing popularity of UV-absorbing or scattering compounds (sunscreens), both independently and as a component of cosmetic products. However, the effectiveness of sunscreen products is directly linked to the efficiency of photo absorption/scattering in the UV range of electromagnetic radiation, in particular, UV-B (280-320 nm) and UV-A (320-400 nm).

Amongst the most widely used combinations of UV-absorbing compounds marketed today is 4-tert-butyl-4-methoxy-dibenzoylmethane (BMDBM) and octyl methoxycinnamate (OMC). Dibenzoylmethane derivatives, including BMDBM, are commonly selected as sunscreen components based on their ability to absorb the full spectrum of UV-A radiation (320-400 nm). Similarly, OMC is known for effectively absorbing electromagnetic radiation in the UV-B range (280-320 nm). However, neither compound alone or in combination is ideal due to their known photo-instability.

Exposure of a dibenzoylmethane derivative to UV-A radiation causes the excitation of an electron in the dibenzoylmethane derivative molecule from an initially occupied, lower energy orbital to a higher energy, previously unoccupied orbital. See Turro, Modern Molecular Photochemistry, 1991. In the electronically excited state, the dibenzoylmethane derivative is prone to degrade via the number of known pathways producing non-UVA-absorbing species and, therefore, can absorb little or no additional UVA energy. Similarly, exposure of OMC to UV radiation transforms the compound into a less absorbing cis-isomer, and thereby less effective at rendering UV absorption. Furthermore, when a dibenzoylmethane derivative (e.g., BMDBM) and OMC are exposed together to UV radiation, a photo reaction, known as the de Mayo reaction, occurs between the species resulting in the rapid disappearance of both species and, consequently, a loss of photoprotective effect.

To photostabilize an electronically excited UV-absorbing organic molecule (e.g., BMDBM), it must be returned to the ground state before it can undergo a photochemical reaction that is destructive to its UV absorbing capabilities.

### SUMMARY

Consequently, a need exists for an effective and safe light absorbing composition that is photochemically stable. Furthermore, a need exists for a compound capable of effectively photostabilizing dibenzoylmethane derivatives (e.g., BMDBM), OMC, or combinations of both.

The present disclosure relates, in some embodiments, to a photostabilized photoactive composition comprising: at least one photoactive compound that develops an excited state when subjected to UV radiation; and at least one para-alkoxyl phenyl substituted propenoic acid derivative of Formula I:

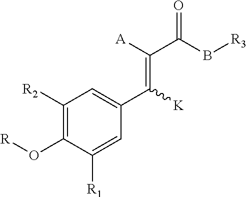

where R is selected from the group consisting of C₁-C₃₀ alkyl; R₁ is selected from the group consisting of C₁-C₁₅ alkoxyl, OH, and H; R₂ is selected from the group consisting of H and C₁-C₁₅ alkoxyl; R₃ is selected from the group consisting of C₁-C₄₀ alkyl; K is selected from the group consisting of C₁-C₁₅ alkyl; B is selected from the group consisting of O and S; and A is selected from the group consisting of CN or (C═O)NR₄(R₅). In some embodiments, R₄ and R₅ may be independently selected from C₁-C₁₅ alkyl, R, R₁, R₂, R₃, R₄, R₅, or any combination thereof may be straight chain structures. In other embodiments, R, R₁, R₂, R₃, R₄, R₅, or any combination thereof may be branched chain structures. At least one para-alkoxy phenyl substituted propenoic acid derivative of Formula I may comprise the Z stereoisomer, the E stereoisomer, or a combination thereof. In some embodiments, at least one para-alkoxyl phenyl substituted propenoic acid derivative of Formula I is operable to quench the excited state energy.

According to some embodiments, a photostabilized photoactive composition may comprise at least one para-alkoxyl phenyl substituted propenoic acid derivative of Formula I that is selected from the group consisting of:

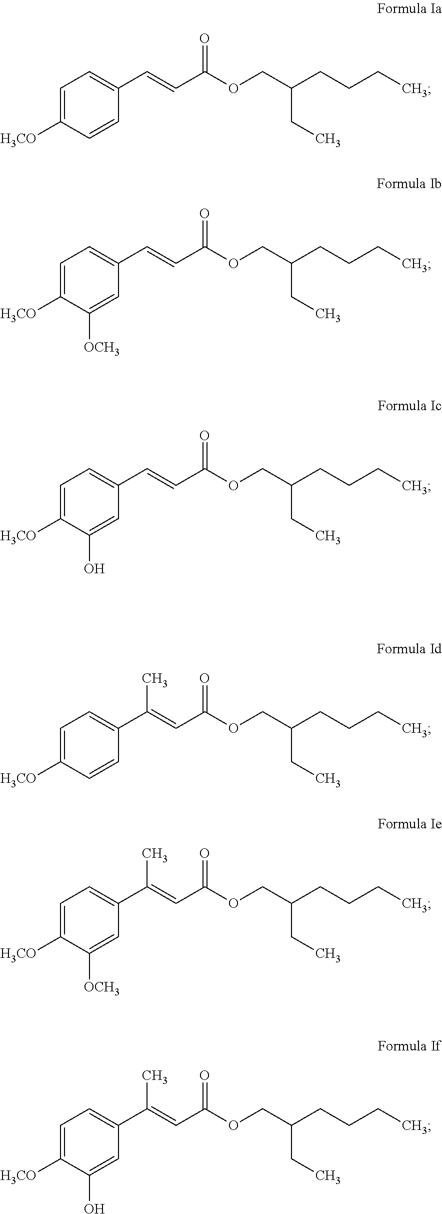 [SMILES: CCCCC(CC)COC(=O)/C=C/c1ccc(OC)cc1 ; CCCCC(CC)COC(=O)/C=C/c1ccc(OC)c(OC)c1 ; CCCCC(CC)COC(=O)/C=C/c1ccc(OC)c(O)c1 ; CCCCC(CC)COC(=O)/C=C(\C)c1ccc(OC)cc1 ; CCCCC(CC)COC(=O)/C=C(\C)c1ccc(OC)c(OC)c1 ; CCCCC(CC)COC(=O)/C=C(\C)c1ccc(OC)c(O)c1]

or
any combination thereof. In some embodiments, at least one para-alkoxy phenyl substituted propenoic acid derivative of Formula I may be present at a concentration of about 0.000001% to about 20%, based on the total weight of the composition. A photostabilized photoactive composition may comprise at least one para-alkoxy phenyl substituted propenoic acid derivative of Formula I present at a concentration comprising about 15% by weight, about 10% by weight, about 5% by weight, about 1% by weight, about 0.5% by weight, about 0.1% by weight, or a combination thereof, based on the total weight of the composition. A photostabilized photoactive composition may comprise at least one para-alkoxyl phenyl substituted propenoic acid derivative of Formula Ia, Ib, Ic, Id, Ie, If, or a combination thereof present at a concentration of about 0.000001% to about 20%, based on the total weight of the composition. According to some embodiments, at least one para-alkoxyl phenyl substituted propenoic acid derivative of Formula Ia, Ib, Ic, Id, Ie, If, or a combination thereof may be present at a concentration, the concentration comprising about 15% by weight, about 10% by weight, about 5% by weight, about 1% by weight, about 0.5% by weight, about 0.1% by weight, or a combination thereof, based on the total weight of the composition.

The present disclosure relates, according to some embodiments, to a photostabilized photoactive composition that may comprise at least one photoactive compound, the at least one photoactive compound comprising 4-methyldibenzoylmethane and derivatives thereof; octyl methoxycinnamate and derivatives thereof; octocrylene and derivatives thereof; p-methoxycinnamic acid esters and derivatives thereof; 1-(4-methoxyphenyl)-3-(4-tert-butylphenyl)propane-1,3-dione and derivatives thereof; oxybenzone and derivatives thereof; bis-ethylhexyloxyphenol methoxyphenyl triazone and derivatives thereof; methylene bis-benzotriazolyl tetramethylbutylphenol and derivatives thereof; 4-methylbenzylidene camphor and derivatives thereof; diethylamino hydroxyl benzoyl hexyl benzoate and derivatives thereof; drometrizole trisiloxane and derivatives thereof; ethylhexyl triazone, diethylhexyl butamido triazone and derivatives thereof; terephthalylidene dicamphor sulfonic acid, its salts, and derivatives thereof, menthyl anthranilate and derivatives thereof; retinol and derivatives thereof; coenzyme Q and derivatives thereof; cholecalciferol and derivatives thereof; porphyrin and derivatives thereof, resveratrol and derivatives thereof; p-aminobenzoic acid, its salts, and derivatives thereof; glyceryl esters; anthranilate and derivatives thereof; cinnamic acid and derivatives thereof, coumarin and derivatives thereof; trihydroxycinnamic acid and derivatives thereof; dibenzalacetone naphtholsulfonate, its salts, and derivatives thereof; dibenzalacetone and derivatives thereof; dihydroxy-naphthoic acid, its salts, and derivatives thereof; o-hydroxydiphenyldisulfonate, its salts, and derivatives thereof; diazole derivatives; quinine derivatives, its salts, and derivatives thereof; quinoline derivatives; hydroxyl-substituted benzophenone derivatives; quinolone derivatives; and combinations of the foregoing. According to some embodiments, a photostabilized photoactive composition may comprise at least one photoactive compound, the at least one photoactive compound comprising benzophenone derivatives; uric acid derivatives; quinine salts; hydroxydiphenyldisulfonate, its salts, and derivatives thereof; tannic acid and derivatives thereof; violuric acid and derivatives thereof; phenylbenzimidazole tetrasulfonate, its salts, and derivatives thereof; terephthalylidene dicamphor sulfonic acid, its salts, and derivatives thereof; benzalacetophenone naphtholsulfonate, its salts, and derivatives thereof; benzoic acid, its salts, and derivatives thereof; o-hydroxydiphenyldisulfonate, its salts, and derivatives thereof; p-naphthalate derivatives; methoxy-substituted uric acid derivatives; hydroquinone, its salts, and derivatives thereof; benzophenone derivatives; dihydroxycinnamic acid, its salts, and derivatives thereof; 1, 3, 5-triazine derivatives; methylene bis-benzotriazolyl tetramethylbutylphenol, its salts, and derivatives thereof; titanium dioxide and derivatives thereof; triazole and derivatives thereof; zinc oxide; bis-ethylhexyloxyphenol methoxyphenyl triazine and its salts; salicylate and derivatives thereof; diethylamino hydroxyl benzoyl hexyl benzoate, its salts, and derivatives thereof; and combinations of the foregoing.

In some embodiments, the present disclosure relates to a photostabilized photoactive composition comprising at least one photoactive compound present at a concentration of about 0.000001% to about 20%, based on the total weight of the composition. At least one photoactive compound may be present at a concentration, the concentration comprising about 15% by weight, about 10% by weight, about 5% by weight, about 1% by weight, about 0.5% by weight, about 0.1% by weight, or a combination thereof, based on the total weight of the composition. In some embodiments, a photostabilized photoactive composition may comprise an oil phase having a dielectric constant of at least about 8. A photostabilized photoactive composition may enhance protection of at least one polymer against UV radiation, the at least one polymer comprising polyvinyl chloride, polystyrene, low-density polyethylene, high-density polyethylene, polyamides, nylon, polypropylene, rubber, and cellulose. According to some embodiments, a photostabilized photoactive composition may enhance protection of at least one coating against UV radiation, the at least one coating comprising adhesives, acrylic paint, latex paint, stains, caulk, sealants, urethanes, enamels, films, and inks. A photostabilized photoactive composition may enhance protection of a sunscreen against UV radiation, wherein application of said sunscreen to a skin of an animal (e.g., human) may protect the skin against UV radiation. In some embodiments, a photostabilized photoactive composition may enhance protection of a cosmetic against UV radiation, wherein application of said cosmetic to a skin or a hair of an animal (e.g., human) may protect the skin or the hair against UV radiation. A photostabilized photoactive composition may comprise a coating, wherein application of said coating to a surface (e.g., polymer, metal, canvas, or wood) may protect the surface against UV radiation.

According to some embodiments, a method of prolonging UV absorption capabilities of a composition containing at least one photoactive compound and at least one para-alkoxyl phenyl substituted propenoic acid derivative, the method comprising adding the at least one para-alkoxyl phenyl substituted propenoic acid derivative of Formula I in an effective amount to the composition, wherein the at least one para-alkoxyl phenyl substituted propenoic acid derivative of Formula I comprises:

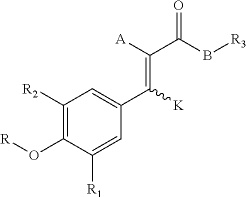

wherein R is selected from the group consisting of C₁-C₃₀ alkyl; R₁ is selected from the group consisting of C₁-C₁₅ alkoxyl, OH, and H; R₂ is selected from the group consisting of H and C₁-C₁₅ alkoxyl; R₃ is selected from the group consisting of C₁-C₄₀ alkyl; K is selected from the group consisting of C₁-C₁₅ alkyl; B is selected from the group consisting of O or S; and A is selected from the group consisting of CN or (C═O)NR₄(R₅), wherein R₄ and R₅ are independently selected from C₁-C₁₅ alkyl; wherein R, R₁, R₂, R₃, R₄, and R₅ may be either straight chain or branched chain; wherein the at least one photoactive compound absorbs UV radiation and thereby protects a substrate from UV radiation; wherein the at least one para-alkoxy phenyl substituted propenoic acid derivative of Formula I comprises the Z stereoisomer, the E stereoisomer, or a combination thereof; and wherein the at least one para-alkoxyl phenyl substituted propenoic acid of Formula I is operable as an excited state energy acceptor.

The present disclosure relates, according to some embodiments, to a method of prolonging UV absorption capabilities of a composition containing at least one photoactive compound and at least one para-alkoxyl phenyl substituted propenoic acid derivative, wherein the composition comprises at least one para-alkoxyl phenyl substituted propenoic acid derivative of Formula I selected from the group consisting of:

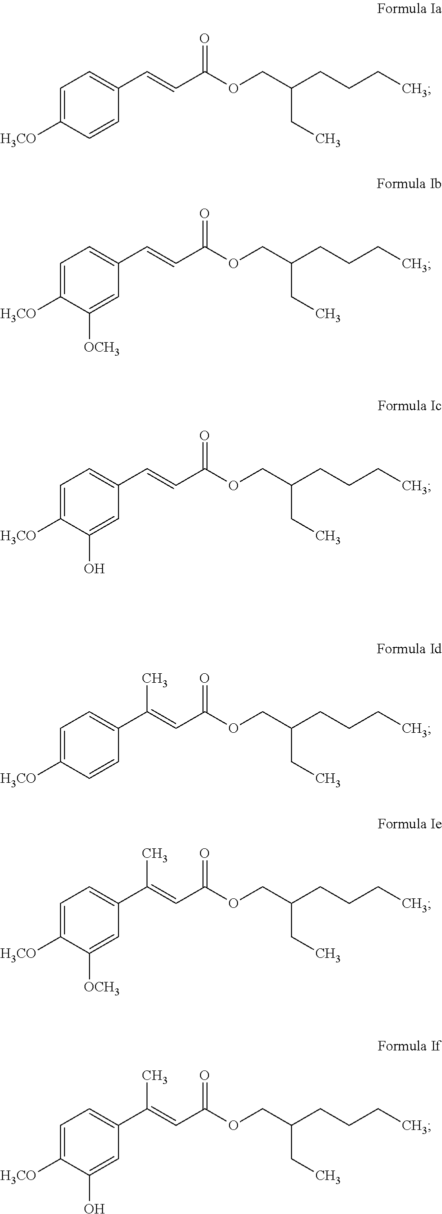 [SMILES: CCCCC(CC)COC(=O)/C=C/c1ccc(OC)cc1 ; CCCCC(CC)COC(=O)/C=C/c1ccc(OC)c(OC)c1 ; CCCCC(CC)COC(=O)/C=C/c1ccc(OC)c(O)c1 ; CCCCC(CC)COC(=O)/C=C(\C)c1ccc(OC)cc1 ; CCCCC(CC)COC(=O)/C=C(\C)c1ccc(OC)c(OC)c1 ; CCCCC(CC)COC(=O)/C=C(\C)c1ccc(OC)c(O)c1]

or
any combination thereof. A method may comprise a substrate, wherein the substrate comprises a polymer, a coating, and a sunscreen. According to some embodiments, a method of prolonging UV absorption capabilities of a composition containing at least one photoactive compound and at least one para-alkoxyl phenyl substituted propenoic acid derivative comprises a polymer, wherein the polymer comprises polyvinyl chloride, polystyrene, low-density polyethylene, high-density polyethylene, polyamides, nylon, polypropylene, rubber, and cellulose.

In some embodiments, a method of prolonging UV absorption capabilities of a composition containing at least one photoactive compound and at least one para-alkoxyl phenyl substituted propenoic acid derivative may comprise a sunscreen, wherein application of said sunscreen to a skin of an animal (e.g., human) thereby protects the skin against UV radiation. A method of prolonging UV absorption capabilities of a composition containing at least one photoactive compound and at least one para-alkoxyl phenyl substituted propenoic acid derivative may comprise a coating, wherein the coating comprises adhesives, acrylic paint, latex paint, stains, caulk, sealants, urethanes, enamels, films, and inks.

According to some embodiments, a method of prolonging UV absorption capabilities of a composition containing at least one photoactive compound and at least one para-alkoxyl phenyl substituted propenoic acid derivative may comprise at least one para-alkoxyl phenyl substituted propenoic acid derivatives of Formula I present at a concentration of about 0.000001% to about 20%, based on the total weight of the composition. In some embodiments, at least one para-alkoxyl phenyl substituted propenoic acid derivative of Formula I is present at a concentration, the concentration comprising about 15% by weight, about 10% by weight, about 5% by weight, about 1% by weight, about 0.5% by weight, about 0.1% by weight, or a combination thereof, based on the total weight of the composition. In some embodiments, at least one para-alkoxyl phenyl substituted propenoic acid derivative of Formula Ia, Ib, Ic, Id, Ie, If, or a combination thereof may be present at a concentration of about 0.000001% to about 20%, based on the total weight of the composition. According to some embodiments, at least one para-alkoxyl phenyl substituted propenoic acid derivative of Formula Ia, Ib, Ic, Id, Ie, If, or a combination thereof, is present at a concentration comprising about 15% by weight, about 10% by weight, about 5% by weight, about 1% by weight, about 0.5% by weight, about 0.1% by weight, or a combination thereof, based on the total weight of the composition.

In some embodiments, the present disclosure relates to at least one photoactive compound that may comprise 4-methyldibenzoylmethane and derivatives thereof, octyl methoxycinnamate and derivatives thereof; octocrylene and derivatives thereof; p-methoxycinnamic acid esters and derivatives thereof; 1-(4-methoxyphenyl)-3-(4-tert-butylphenyl)propane-1,3-dione and derivatives thereof; oxybenzone and derivatives thereof; bis-ethylhexyloxyphenol methoxyphenyl triazone and derivatives thereof; methylene bis-benzotriazolyl tetramethylbutylphenol and derivatives thereof; 4-methylbenzylidene camphor and derivatives thereof; diethylamino hydroxyl benzoyl hexyl benzoate and derivatives thereof; drometrizole trisiloxane and derivatives thereof; ethylhexyl triazone, diethylhexyl butamido triazone and derivatives thereof; terephthalylidene dicamphor sulfonic acid, its salts, and derivatives thereof, menthyl anthranilate and derivatives thereof; retinol and derivatives thereof; coenzyme Q and derivatives thereof; cholecalciferol and derivatives thereof; porphyrin and derivatives thereof, resveratrol and derivatives thereof; p-aminobenzoic acid, its salts, and derivatives thereof; glyceryl esters; anthranilate and derivatives thereof; cinnamic acid and derivatives thereof, coumarin and derivatives thereof; trihydroxycinnamic acid and derivatives thereof; dibenzalacetone naphtholsulfonate, its salts, and derivatives thereof; dibenzalacetone and derivatives thereof; dihydroxy-naphthoic acid, its salts, and derivatives thereof; o-hydroxydiphenyldisulfonate, its salts, and derivatives thereof; diazole derivatives; and combinations of the foregoing. In some embodiments, at least one photoactive compound may comprise quinine derivatives, its salts, and derivatives thereof; quinoline derivatives; hydroxyl-substituted benzophenone derivatives; quinolone derivatives; benzophenone derivatives; uric acid derivatives; quinine salts; hydroxydiphenyldisulfonate, its salts, and derivatives thereof; tannic acid and derivatives thereof; violuric acid and derivatives thereof; phenylbenzimidazole tetrasulfonate, its salts, and derivatives thereof; terephthalylidene dicamphor sulfonic acid, its salts, and derivatives thereof; benzalacetophenone naphtholsulfonate, its salts, and derivatives thereof; benzoic acid, its salts, and derivatives thereof; o-hydroxydiphenyldisulfonate, its salts, and derivatives thereof; p-naphthalate derivatives; methoxy-substituted uric acid derivatives; hydroquinone, its salts, and derivatives thereof; benzophenone derivatives; dihydroxycinnamic acid, its salts, and derivatives thereof; 1, 3, 5-triazine derivatives; methylene bis-benzotriazolyl tetramethylbutylphenol, its salts, and derivatives thereof; titanium dioxide and derivatives thereof; triazole and derivatives thereof; zinc oxide; bis-ethylhexyloxyphenol methoxyphenyl triazine and its salts; salicylate and derivatives thereof; diethylamino hydroxyl benzoyl hexyl benzoate, its salts, and derivatives thereof; and combinations of the foregoing.

According to some embodiments, a method of prolonging UV absorption capabilities of a composition containing at least one photoactive compound and at least one para-alkoxyl phenyl substituted propenoic acid derivative may comprise at least one photoactive compound present at a concentration of about 0.000001% to about 20%, based on the total weight of the composition. At least one photoactive compound may be present at a concentration comprising about 15% by weight, about 10% by weight, about 5% by weight, about 1% by weight, about 0.5% by weight, about 0.1% by weight, or a combination thereof, based on the total weight of the composition. According to some embodiments, a composition may include an oil phase having a dielectric constant of at least about 7, and in some embodiments, a dielectric constant of at least about 8. In some embodiments, a method may comprise a cosmetic, wherein application of said cosmetic to a skin or a hair of an animal (e.g., human) thereby protects the skin or the hair against UV radiation. According to some embodiments, a method comprises a coating, wherein application of said coating to a surface (e.g., polymer, metal, canvas, or wood) thereby protects the surface against UV radiation.

### BRIEF DESCRIPTION OF THE DRAWINGS

Some embodiments of the disclosure may be understood by referring, in part, to the present disclosure and the accompanying drawings, wherein:

FIG. 1 is a graph illustrating the significant change in the UV absorption spectra and decrease in UVA absorption when a composition comprising BMDBM and OMC according to an embodiment of the disclosure were irradiated with UV light;

FIG. 2 is a graph illustrating the UV absorption spectra when a composition comprising BMDBM, OMC, and APP according to an embodiment of the disclosure were irradiated with UV light; and

FIG. 3 is a graph illustrating the amount of BMDBM remaining in a composition according to an embodiment of the disclosure after irradiation with UV light.

### DETAILED DESCRIPTION

The present disclosure relates, in some embodiments, to para-alkoxyl phenyl substituted propenoic acid (APP) derivatives capable of resolving excited energy states of certain photo unstable chromophores. Particularly, APP derivatives accept energy from electronically excited UV-absorbing chromophore molecules, such as dibenzoylmethane derivatives, such that an excited state of these UV-absorbing molecules is photostabilized (i.e., returned to a ground state). Upon photostabilization, a UV-absorbing chromophore molecule is preserved and may be capable of absorbing additional UV radiation. Accordingly, enhanced protection of surfaces from UV radiation may be achieved by applying compositions comprising one or more APP analogue compound together with UV-absorbing materials (e.g., dibenzoylmethane derivatives). In some embodiments, a cosmetic, a sunscreen, a polymer (e.g., polyethylene), and/or a coating (e.g., paints) may comprise at least one APP analogue and at least one UV-absorbing compound, wherein applying said cosmetic, sunscreen, polymer, and/or coating to a surface (e.g., skin, hair, metal, plastic, wood) may protect the applied surface from UV radiation.

This disclosure relates to sunscreen, cosmetic, polymer, coating, and dermatological compositions comprising a mixture of at least one photoactive compound (e.g., BMDBM) and at least one APP derivative that functions as an excited state energy acceptor. This disclosure further relates to methods of prolonging UV absorption capabilities of a composition comprising UV-absorbing materials using at least one APP analogue that may function as an excited state energy acceptor.

The present disclosure relates to photostabilized photoactive compositions. Such compositions may comprise one or more photoactive compound that develops an excited state when subjected to UV radiation. Photostabilized photoactive compositions may also comprise an excited state energy acceptor comprising one or more APP derivatives of Formula I. According to this disclosure APP derivatives of Formula I are capable of photostabilizing photoactive compositions by serving as excited state energy acceptors. However, this disclosure is not limited to any particular mechanism by which APP derivatives of Formula I are capable of photostabilizing photoactive compositions.

In some embodiments an APP analogue may comprise a structure according to Formula I:

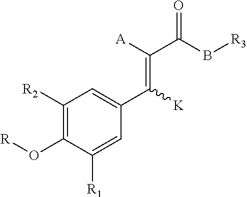

In some embodiments R is selected from a group consisting of C₁-C₃₀ alkyl (straight chain or branched chain). For example, R may comprise a methyl, ethyl, isopropyl, n-butyl, sec-butyl, or tert-butyl group. In some embodiments, R is a hydrogen. Without being limited to any particular chemical mechanism of action, hydration of a phenolic hydroxyl group when R is a hydrogen may lead to an extended lifetime of an excited state. Such extended excited state may result in formation of radicals and potential phototoxicity and/or photodegradation. Further, an alkoxyl substitution at a para position may be critical and may allow for a shorter lifetime of the electronically excited states of these molecules.

According to some embodiments, R₁ is selected from a group consisting of C₁-C₁₅ alkoxyl (straight chain or branched chain), OH, or H. For example, R₁ may include, but is not limited to methoxyl, ethoxyl, OH, or H. In some embodiments, R₂ is selected from a group consisting of H or C₁-C₁₅ alkoxyl (straight chain or branched chain). For example, R₂ may include, but is not limited to methoxyl, ethoxyl, OH, or H. In some embodiments R₃ is selected from a group consisting of C₁-C₄₀ alkyl (straight chain or branched chain). For example, R₃ may include, but is not limited to methyl, ethyl, propyl, butyl, pentyl, hexyl, or octyl. In some embodiments, the APP derivatives of Formula I comprise the Z stereoisomer, the E stereoisomer, or combinations thereof.

In some embodiments B is selected from a group consisting of O or S. In some embodiments, K is selected from the group consisting of C₁-C₁₅ alkyl. Such selections may provide a greater ability by a compound to accept energy and/or have an excited state with a shorter lifetime.

In some embodiments, A may be selected from a group consisting of CN or (C═O)NR₄(R₅), wherein R₄ and R₅ are independently selected from C₁-C₁₅ alkyl (straight chain or branched chain). For example, R₄ and R₅ can include, but are not limited to methyl, ethyl, propyl, butyl. According to some embodiments of the disclosure, A does not include (C═O)O-alkyl. A material comprising a (C═O)O-alkyl substituent at A may be thermodynamically unstable under basic conditions. In some embodiments, compounds of Formula I are selected from: Formula Ia; Formula Ib; Formula Ic; Formula Id; Formula Ie; Formula If; or any combination thereof.

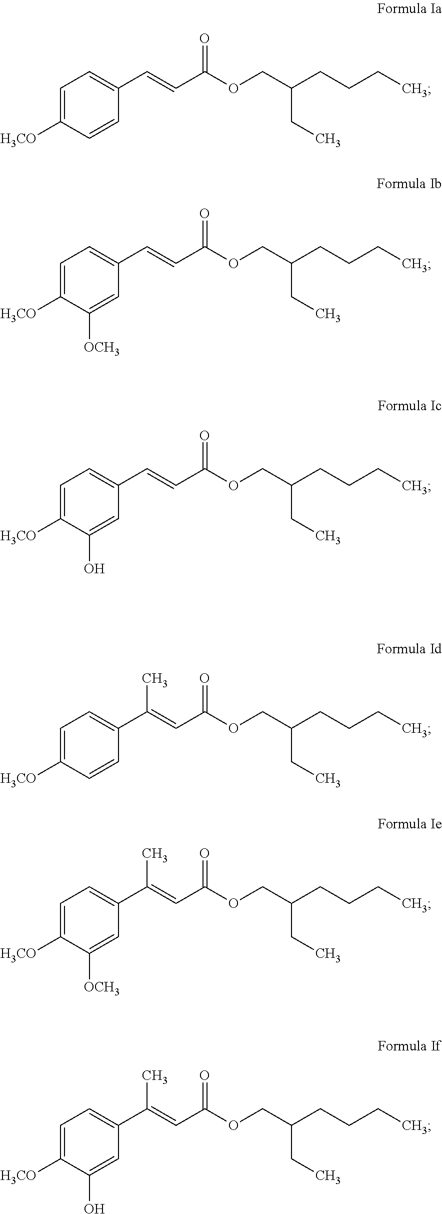 [SMILES: CCCCC(CC)COC(=O)/C=C/c1ccc(OC)cc1 ; CCCCC(CC)COC(=O)/C=C/c1ccc(OC)c(OC)c1 ; CCCCC(CC)COC(=O)/C=C/c1ccc(OC)c(O)c1 ; CCCCC(CC)COC(=O)/C=C(\C)c1ccc(OC)cc1 ; CCCCC(CC)COC(=O)/C=C(\C)c1ccc(OC)c(OC)c1 ; CCCCC(CC)COC(=O)/C=C(\C)c1ccc(OC)c(O)c1]

or

The present disclosure, in some embodiments, may comprise derivatives of Formula I that may stabilize photoactive compounds that develop excited states when subjected to UV radiation. In some embodiments, photoactive compounds include dibenzoyl methane derivatives such as: 2-methyldibenzoylmethane; 4-methyldibenzoylmethane; 4-isopropyldibenzoylmethane; 4-tert-butyldibenzoylmethane; 2,4-dimethydibenzoylmethane; 2-5-dimethyl dibenzoylmethane; 4,4′-diispropyl dibenzoylmethane; 4,4′-dimethoxy dibenzoylmethane; 4-tert-butyl-4′-methoxy dibenzoylmethane; 2-methyl-5-isopropyl-4′-methoxydibenzoylmethane; 2-methyl-5-tert-butyl-4′-methoxydibenzoylmethane; 2,4-dimethyl-4′-methoxydibenzoylmethane; and 2,6-dimethyl-4-tert-butyl-4′-methoxydibenzoylmethane.

In some embodiments photoactive compounds may be alternatively described as UV-absorbing compounds and/or UV filtering compounds. UV-absorbing compounds may include octyl methoxycinnamate (OMC), octocrylene, salicylic acid esters (e.g., ethylhexyl salicylate and homomenthyl salicylate), p-methoxycinnamic acid esters (e.g., ethylhexyl methoxycinnamate and Isoamyl methoxycinnamate), avobenzone (also known as butyl methoxydibenzoylmethane), oxybenzone, bis-ethylhexyloxyphenol methoxyphenyl triazone, methylene bis-benzotriazolyl tetramethylbutylphenol, 4-methylbenzylidene camphor, diethylamino hydroxyl benzoyl hexyl benzoate, drometrizole trisiloxane, ethylhexyl triazone, diethylhexyl butamido triazone, terephthalylidene dicamphor sulfonic acid and its salts, and menthyl anthranilate.

In some embodiments, photoactive compounds that develop excited states when subjected to UV radiation may comprise p-aminobenzoic acid, its salts, and its derivatives (ethyl, isobutyl, glyceryl esters; p-dimethylaminobenzoic acid); anthranilates (o-aminobenzoates; methyl, menthyl, phenyl, benzyl, phenylethyl, linalyl, terpinyl, and cyclohexenyl esters); salicylates (octyl, amyl, phenyl, benzyl, (+)-menthyl, (−)-menthyl, glyceryl, and dipropyleneglycol esters); cinnamic acid derivatives ((+)-menthyl, (−)-menthyl, and benzyl esters, alpha-phenyl cinnamonitrile; butyl cinnamoyl pyruvate); coumarin derivatives umbelliferone, methylumbelliferone, methylaceto-umbelliferone); camphor derivatives (3 benzylidene, 4-methylbenzylidene, polyacrylamidomethyl benzylidene, benzalkonium methylsulfate, benzylidene camphor sulfonic acid, and terephthalylidene dicamphor sulfonic acid); trihydroxycinnamic acid derivatives (esculetin, methylesculetin, daphnetin, and the glucosides, esculin and daphnin); hydrocarbons (diphenylbutadiene, stilbene); dibenzalacetone; benzalacetophenone; naphtholsulfonates (sodium salts of 2-naphthol-3,6-disulfonic and of 2-naphthol-6,8-disulfonic acids); dihydroxy-naphthoic acid and its salts; o- and p-hydroxydiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinolone derivatives (8-hydroxyquinoline salts, 2-phenylquinoline); hydroxy- or methoxy-substituted benzophenones; uric acid derivatives; violuric acid derivatives; tannic acid and its derivatives; hydroquinone; benzophenones (oxybenzone, sulisobenzone, dioxybenzone, benzoresorcinol, octabenzone, 4-isopropyldibenzoylmethane, butyl methoxydibenzoylmethane, etocrylene, and 4-isopropyl-dibenzoylmethane); 2-ethylhexyl p-methoxycinnamate; 4,4′-t-butyl methoxydibenzoylmethane; octyldimethyl p-aminobenzoate; digalloyl trioleate; ethyl 4-[bis(hydroxypropyl)]aminobenzoate; 2-ethylhexyl salicylate; glycerol p-aminobenzoate; 3,3,5-trimethylcyclohexylsalicylate; and combinations thereof.

Photoactive compositions may include at least one UV-A (320-400 nm) photoactive compound and at least one UV-B (280-320 nm) photoactive compound. According to some embodiments, photoactive compounds that develop excited states when subjected to UV radiation may comprise 4-methyldibenzoylmethane and derivatives thereof; octyl methoxycinnamate and derivatives thereof; octocrylene and derivatives thereof; p-methoxycinnamic acid esters and derivatives thereof; 1-(4-methoxyphenyl)-3-(4-tert-butylphenyl)propane-1,3-dione and derivatives thereof; oxybenzone and derivatives thereof; bis-ethylhexyloxyphenol methoxyphenyl triazone and derivatives thereof; methylene bis-benzotriazolyl tetramethylbutylphenol and derivatives thereof; 4-methylbenzylidene camphor and derivatives thereof; diethylamino hydroxyl benzoyl hexyl benzoate and derivatives thereof; drometrizole trisiloxane and derivatives thereof; ethylhexyl triazone, diethylhexyl butamido triazone and derivatives thereof; terephthalylidene dicamphor sulfonic acid, its salts, and derivatives thereof, menthyl anthranilate and derivatives thereof; retinol and derivatives thereof; coenzyme Q and derivatives thereof; cholecalciferol and derivatives thereof; porphyrin and derivatives thereof, resveratrol and derivatives thereof; p-aminobenzoic acid, its salts, and derivatives thereof; glyceryl esters; anthranilate and derivatives thereof; cinnamic acid and derivatives thereof, coumarin and derivatives thereof; trihydroxycinnamic acid and derivatives thereof; dibenzalacetone naphtholsulfonate, its salts, and derivatives thereof; dibenzalacetone and derivatives thereof; dihydroxy-naphthoic acid, its salts, and derivatives thereof; o-hydroxydiphenyldisulfonate, its salts, and derivatives thereof; diazole derivatives; quinine derivatives its, salts, and derivatives thereof; quinoline derivatives; hydroxyl-substituted benzophenone derivatives; quinolone derivatives; benzophenone derivatives; uric acid derivatives; quinine salts; hydroxydiphenyldisulfonate, its salts, and derivatives thereof; tannic acid and derivatives thereof; violuric acid and derivatives thereof; phenylbenzimidazole tetrasulfonate, its salts, and derivatives thereof; terephthalylidene dicamphor sulfonic acid, its salts, and derivatives thereof; benzalacetophenone naphtholsulfonate, its salts, and derivatives thereof; benzoic acid, its salts, and derivatives thereof; o-hydroxydiphenyldisulfonate, its salts, and derivatives thereof; p-naphthalate derivatives; methoxy-substituted uric acid derivatives; hydroquinone, its salts, and derivatives thereof; benzophenone derivatives; dihydroxycinnamic acid, its salts, and derivatives thereof; 1, 3, 5-triazine derivatives; methylene bis-benzotriazolyl tetramethylbutylphenol, it salts and derivatives thereof; titanium dioxide and derivatives thereof; triazole and derivatives thereof; zinc oxide; bis-ethylhexyloxyphenol methoxyphenyl triazine and its salts; salicylate and derivatives thereof; diethylamino hydroxyl benzoyl hexyl benzoate, its salts, and derivatives thereof; and combinations of the foregoing.

According some embodiments, photoactive compounds that develop excited states when subjected to UV radiation may comprise aminobenzoic acid, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate; zinc oxide, diethanolamine methoxycinnamate, ethyl-[bis(hydroxypropyl)]aminobenzoate, glyceryl aminobenzoate, 4-isopropyl dibenzoylmethane, 4-methylbenzylidene camphor, terephthalylidene dicamphor sulfonic acid, sulisobenzone, camphor benzalkonium methosulfate, homosalate, benzophenone, terephthalydene dicamphor sulfonic acid, benzylidene camphor sulfonic acid, polyacrylamidomethyl benzylidene camphor, PEG-25 PABA, isoamyl p-methoxycinnamate, ethylhexyl triazone, drometrizole trisiloxane, diethylhexyl butamido triazone, 4-methylbenzylidene camphor, benzylidene camphor, ethylhexyl salicylate, ethylhexyl dimethyl PABA, methylene bis benztriazolyl tetramethylbutylphenol, disodium phenyl dibenzimidazole tetrasulfonate, bis ethylhexyloxyphenol methoxyphenol triazine, methylene bisbenzotriazolyl tetramethylbutylphenol, 4-(2-oxo-3-bornylidenemethyl)benzene-sulfonic acid, 2-methyl-5-(2-oxo-3-bornylidenemethyl)sulfonic acid and the salts thereof, 2-(4′-diethylamino-2′-hydroxybenzoyl) benzoic acid hexyl ester, 2,2′-methylenebis(6-(2H-benzotriazol-2-yl)-4-(1,1,3,3-tetramethylbutyl)phenol), bisoctyltriazole, diethylhexyl 2,6-naphthalate, and bisethylhexyloxyphenol methoxyphenyl triazine.

A photoactive compound may be considered stable when, for example, after about 30 Minimum Erythemal Dose (“MED”) irradiation, the photoactive compound has retained at least about 90% of its original absorbance at a wavelength, or over a range of wavelengths of interest (e.g., the wavelength at which a photoactive compound has a peak absorbance, such as 350-370 nm for Avobenzone). In some embodiments, a sunscreen, a cosmetic, a polymer, a coating, or a dermatological composition may include a plurality of photoactive compounds and may be considered stable as a whole when, for example, after about 30 MED irradiation, the sunscreen, cosmetic, or dermatological composition has retained at least about 90% of its original absorbance at one or more wavelengths of interest (e.g., at or near the peak absorbance wavelength of the primary photoactive compound). According to some embodiments, a composition comprising an effective amount of at least one APP derivative may include a concentration of at least one APP derivative, wherein after about 30 MED irradiation, the composition retains at least about 90% of its original absorbance at one or more wavelengths of interest.

The present disclosure, in some embodiments may comprise at least one APP analogue according to Formula I, Ia, Ib, Ic, Id, Ie, If, or a combination thereof, wherein at least one APP analogue may function as an excited state energy acceptor.

In accordance with some embodiments, APP derivatives of formula I, Ia, Ib, Ic, Id, Ie, If, or a combination thereof are included in a sunscreen, cosmetic, or dermatological composition for coating a skin surface to protect skin from UV damage when exposed to sunlight, or other UV radiation. In some embodiments, sunscreen, cosmetic, or dermatological compositions include a UV-A filter and/or a UV-B filter compound and/or a broad-band filter compound, particularly a dibenzoylmethane derivative UV-A filter that is photostabilized by at least one APP compound for protection of skin from UV-A and/or UV-B wavelength radiation.

The present disclosure, in some embodiments may comprise at least one APP compound according to Formula I, Ia, Ib, Ic, Id, Ie, If, or a combination thereof is present at a concentration of about 0.000001% to about 20%, based on the total weight of the composition. In some embodiments, the concentration of at least one APP compound is present at a concentration comprising about 15% by weight, about 10% by weight, about 5% by weight, about 1% by weight, about 0.5% by weight, about 0.1% by weight, or a combination thereof, based on the total weight of the composition. In some embodiments, at least one para-alkoxyl phenyl substituted propenoic acid derivative of Formula I is present at a concentration of about 1% to about 3%, based on the total weight of the composition.

In some embodiments, a sunscreen, cosmetic, polymer, coating, or dermatological composition may comprise at least one APP compound according to Formula I, Ia, Ib, Ic, Id, Ie, If, or a combination thereof present at a concentration of about 0.000001% to about 20%, based on the total weight of the composition. In some embodiments, the concentration of at least one APP compound is present at a concentration comprising about 15% by weight, about 10% by weight, about 5% by weight, about 1% by weight, about 0.5% by weight, about 0.1% by weight, or a combination thereof, based on the total weight of the composition. In some embodiments, at least one para-alkoxyl phenyl substituted propenoic acid derivative of Formula I is present at a concentration of about 1% to about 3%, based on the total weight of the composition.

The present disclosure, in some embodiments may comprise at least one photoactive compound as described herein present at a concentration of about 0.000001% to about 20%, based on the total weight of the composition. In some embodiments, the at least one photoactive compound is present at a concentration comprising about 15% by weight, about 10% by weight, about 5% by weight, about 1% by weight, about 0.5% by weight, about 0.1% by weight, or a combination thereof, based on the total weight of the composition.

In some embodiments, a sunscreen, cosmetic, polymer, coating, or dermatological composition may comprise at least one photoactive compound as described herein present at a concentration of about 0.000001% to about 20%, based on the total weight of the composition. In some embodiments, the concentration of at least one photoactive compound is present at a concentration comprising about 15% by weight, about 10% by weight, about 5% by weight, about 1% by weight, about 0.5% by weight, about 0.1% by weight, or a combination thereof, based on the total weight of the composition.

In some embodiments, APP derivatives may be hydrophobic and may not be water soluble. Waterproof sunscreen, cosmetic, or dermatological compositions may comprise at least one hydrophobic APP analogue. In some embodiments, compositions and methods may comprise an oil phase. An oil phase may further comprise at least one polar solvent, wherein at least one polar solvent may increase the polarity of an oil phase of a composition including APP derivatives. In some embodiments, increasing the polarity of an oil phase of a composition may increase the photostability of photoactive compounds in a composition. In some embodiments, at least one polar solvent is present in the oil-phase of the composition, wherein the amount of polar solvent present may increase the dielectric constant of the oil-phase of the composition to a dielectric constant of at least about 7. Enough polar solvent may be present to raise the dielectric constant of the oil-phase of the composition to at least about 8. In some embodiments, polar solvents comprise acetone, ethanol, n-butanol, isopropanol, acetonitrile, ethyl acetate, dimethyl sulfoxide, water, and combinations thereof.

According to some embodiments, a photoactive composition may include one or more photoactive compounds, wherein the photoactive compound(s) act to absorb UV radiation and thereby protect the substrate (e.g., human skin, animal skin, resins, films, polymers, metals, glass, paints, wood) from the effects of UV radiation. The absorption process causes a photoactive compound to reach an excited state, wherein the excited state is characterized by the presence of excited electronic energy (e.g., singlet state energy or triplet state energy), as compared to the ground state of the photoactive compound. Once a photoactive compound reaches an excited state, there exists a number of pathways by which the excited photoactive compound can dissipate its excess energy (e.g., singlet and/or triplet energy), however, many of those pathways adversely affect the ability of the photoactive compound to further absorb UV radiation if they result in structural rearrangement of the photoactive compound (e.g., de Mayo reaction and fragmentation). APP derivatives may accept electronic excited state energy from UV absorbers, providing photostability to the photoactive compound. In some embodiments, APP derivatives provide electronic excited state energy quenching of UV-absorbing compounds in compositions, the compositions comprising sunscreen, cosmetic, polymer, coating, and dermatological formulations.

In some embodiments, the sunscreen, cosmetic, or dermatological compositions described herein may comprise one or more APP derivatives and one or more photoactive compounds. Compositions may include both UV-A and UV-B photoactive compounds in a cosmetically acceptable carrier, optionally including additives, such as emollients, stabilizers, emulsifiers, and combinations thereof. Additives may be used in preparing a sunscreen, cosmetic, or dermatological composition in an emulsion (oil-in-water or water-in-oil) from a composition that includes one or more photoactive compounds and a solvent or a solvent combination that includes one or more organic solvents and water. An emulsion may be an oil-in-water emulsion, wherein the oil phase is primarily formed from a mixture of the UV filter compound(s), and may include a dibenzoylmethane derivative, such as Avobenzone, and one or more organic solvents.

In some embodiments, a composition comprising at least one APP derivative and at least one photoactive compound may be used for sunscreen, cosmetic, or dermatological compositions, wherein compositions treat, care, decorate, and/or cleanse skin and/or hair from an animal (e.g., human). Sunscreen, cosmetic, or dermatological compositions may comprise cosmetic auxiliaries such as those conventionally used in such preparations, such as preservatives, bactericides, perfumes, antifoaming agents, dyes, pigments which have a coloring effect, thickeners, moisturizers and/or humectants, fats, oils, waxes or other conventional constituents of a sunscreen, cosmetic, or dermatologic formulation, other conventional constituents comprising alcohols, polyols, polymers, foam stabilizers, electrolytes, organic solvents, silicone derivatives, or combinations thereof.

In some embodiments, a composition comprising at least one APP derivatives and at least one photoactive compound may be used for providing UV-A and UV-B radiation protection for natural and synthetic polymers, the natural and synthetic polymers comprising polyvinyl chloride, polystyrene, low-density polyethylene, high-density polyethylene, polyamides, nylon, polypropylene, rubber, and cellulose. Furthermore, natural and synthetic polymers may crack, discolor, and/or disintegrate due to UV radiation induced damage. In some embodiments, compositions may provide UV-A and UV-B radiation protection for a coating, the coating comprising adhesives, acrylic paint, latex paint, stains, caulk, sealants, urethanes, enamels, films, and inks.

As will be understood by those skilled in the art who have the benefit of the instant disclosure, other equivalent or alternative compositions and methods for formulating a photostable UV absorbing composition comprising para-alkoxyl phenyl substituted propenoic acid (APP) derivatives can be envisioned without departing from the description contained herein. Accordingly, the manner of carrying out the disclosure as shown and described is to be construed as illustrative only.

Persons skilled in the art may make various changes in the identity and quantity of components without departing from the scope of the instant disclosure. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Persons skilled in the art may make various changes in methods of preparing and using a composition of the disclosure. For example, a composition, device, and/or system may be prepared and or used as appropriate for animal and/or human use (e.g., with regard to sanitary, infectivity, safety, toxicity, biometric, and other considerations). Elements, compositions, devices, systems, methods, and method steps not recited may be included or excluded as desired or required.

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value+/−about 10%, depicted value+/−about 50%, depicted value+/−about 100%) and/or a range endpoint. With respect to the former, a value of 50 depicted in an example, table, and/or drawing may form the basis of a range of, for example, about 45 to about 55, about 25 to about 100, and/or about 0 to about 100. Disclosed percentages are weight percentages except where indicated otherwise.

These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

The title, abstract, background, and headings are provided in compliance with regulations and/or for the convenience of the reader. They include no admissions as to the scope and content of prior art and no limitations applicable to all disclosed embodiments.

### EXAMPLES

Comparisons were performed to evaluate the abilities of APP derivatives of Formula I, Ia, Ib, Ic, Id, Ie, If, or combination thereof to photostabilize photoactive compounds. UV light exposure is often measured in MED rather than a measurement of time. MED relates to the minimal dosage of UV exposure required to result in redness 24 hours after exposure. Although technically an individualized measurement that would vary depending on the sensitivity of an individual's skin, MED measurements have been standardized with the advent of UV bulbs and lamps.

### Example 1

### Comparing the Photostability of a Composition Containing BMDBM and OMC

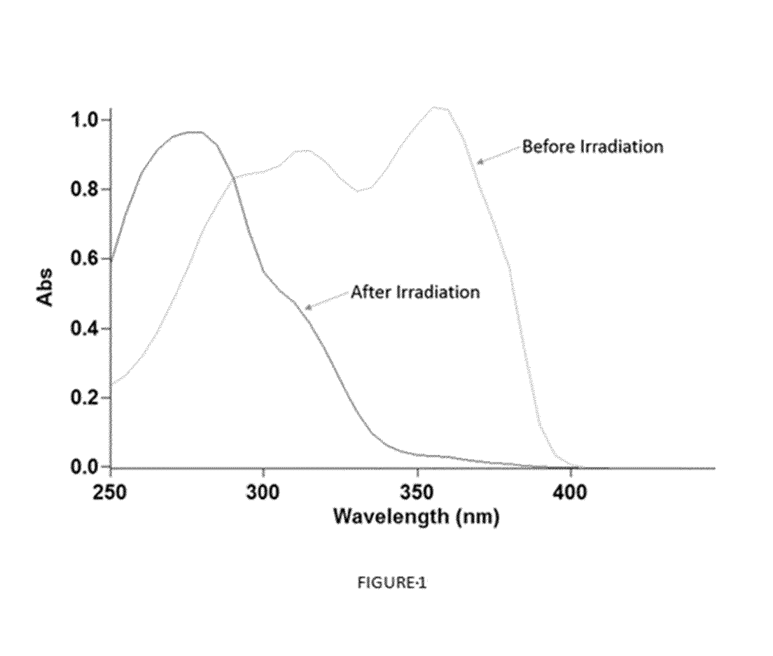

FIG. 1 illustrates the photoinstability of a composition containing BMDBM and OMC. As shown in FIG. 1, a composition containing an acetonitrile solution including 10 ppm BMDBM and 10 ppm OMC was irradiated with 0 MED (before irradiation line) and 15 MED (after irradiation line) of UV light. Absorption was measured for wavelengths 250 nm to 500 nm in length. BMDBM absorbs wavelengths around 375 nm in length, which can be seen as a distinct peak on the line illustrating the composition which was not irradiated with UV light. However, as shown in FIG. 1, after 15 MED UV irradiation, the BMDBM peak at 375 nm is substantially diminished. Thus, FIG. 1 illustrates that the BMDBM in the composition had a drastically reduced capability of absorbing light after exposure to 15 MED UV irradiation.

### Example 2

### Photostabilization of a Photoactive Composition with an APP Analogue

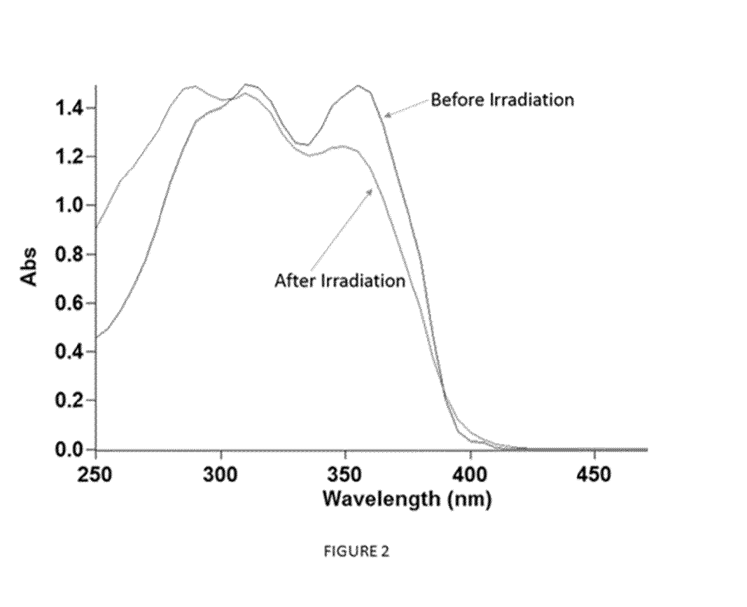

FIG. 2 illustrates the photostabilization of a photoactive composition by the addition of an APP analogue of Formula I. As shown in FIG. 2, a composition containing an acetonitrile solution of 10 ppm BMDBM, 10 ppm of OMC, and 5 ppm of an APP analogue of Formula I was irradiated with 0 MED (Before irradiation line) and 15 MED (After irradiation line) of UV light. The APP analogue was a 1:1 blend of Formula Ia and Formula Ib. As shown in FIG. 2, the BMDBM peak at 375 nm is only moderately diminished after exposure of the composition to 15 MED (blue line). Thus, FIG. 2 illustrates that the APP analogue of Formula I had a photostabilizing effect on the composition.

### Example 3

### Comparing the Stability of Compositions with and without APP

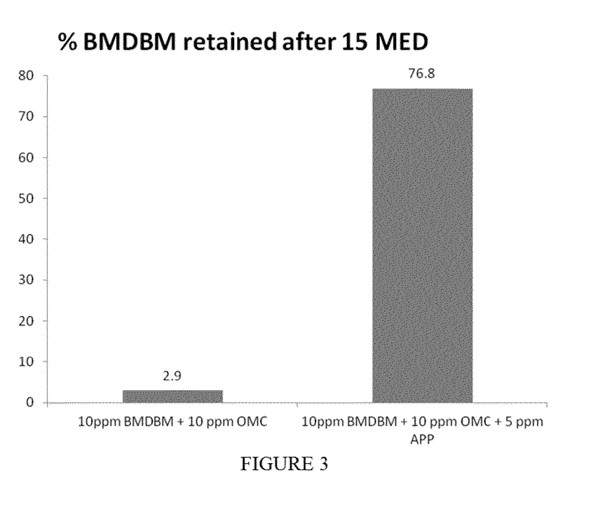

FIG. 3 illustrates the percentage of BMDBM remaining in compositions after irradiation with 15 MED of UV light. As shown in FIG. 3, only 2.9% of the BMDBM of a composition containing 10 ppm BMDBM and 10 ppm OMC remained capable of absorbing light at 375 nm after being irradiated with 15 MED of UV light. By contrast, approximately 76.8% of the BMDBM of a composition containing 10 ppm BMDBM, 10 ppm OMC, and 5 ppm of an APP analogue of Formula I retained the ability to absorb light at 375 nm. The APP analogue was a 1:1 blend of Formula Ia and Formula Ib. Thus, as illustrated in FIG. 3, APP derivatives of Formula I are capable of photostabilizing photoactive compositions.

### Example 4

### Reaction of Acetophenone with Methyl Cyanoacetate

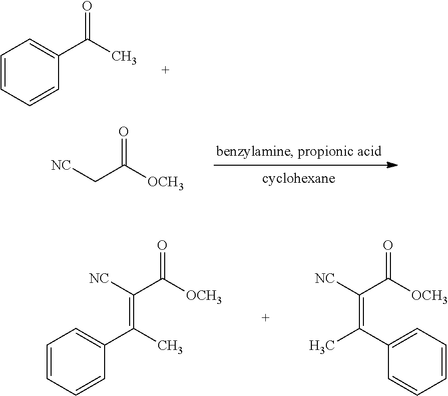

Acetophenone (1200 g, 10 mol) and methyl cyanoacetate (1000 g, 10 mol) were added to each other in cyclohexane (600 mL), in which a heterogeneous solution was obtained. Then benzyl amine (40 g) and propionic acid (60 g) were added to the mixture in one turn. The mixture was stirred at reflux for 15 h and the water formed was removed en vacuo through an azeotrope with cyclohexane until no more water was formed. The remaining cyclohexane was distilled off and then mixture was cooled down, the mixture was washed with water (1000 mL×3) and brine, then dried with CaCl₂ and evaporated to dryness. The residue was distilled under reduced pressure to obtain 1108 g of pure product with a yield of 55.1% (mp=120° C./1 mmHg) and 760 g of a mixture of acetophenone and methyl cyanoacetate was recovered.

### Example 5

### Reaction of 4-Methoxyacetophenone with Methyl Cyanoacetate

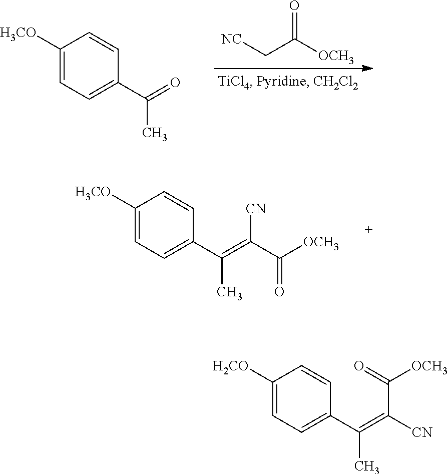

4-Methoxyacetophenone (375 g, 2.5 mol) and methyl cyanoacetate (320 g, 3.25 mol) were dissolved in CH₂Cl₂ (2500 mL). TiCl₄ (710 g, 3.75 mol) was first added at 30° C. for a period of 1 h, then a solution of pyridine (400 g, 5 mol) dissolved in CH₂Cl₂ (400 mL) were added slowly over a period of 0.5 h with a gentle reflux. Then the mixture was heated and stirred at reflux for 0.5 h (monitored by TLC). Another part of TiCl₄ (110 g) and pyridine (60 g) were added to the mixture in turn. Then the mixture was stirred under reflux (monitored by TLC). TLC showed that 4-methoxyacetophenone was consumed completely, the mixture was poured into water (2000 mL) and stirred sufficiently to transparent liquid. The organic phase separated was washed with water (1000 mL×3) and brine, then dried with Na₂SO₄ and evaporated to dryness. The residue was distilled under reduced pressure to obtain 350 g of pure product with a yield of 60.5% (mp=160° C./1 mmHg).

### Example 6

### Synthetic Route 1 for Reaction of 3,4-Dimethoxyacetophenone with Methyl Cyanoacetate

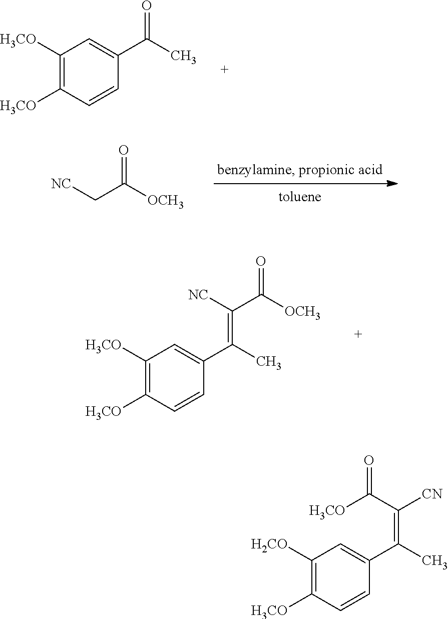

3,4-dimethoxyacetophenone (720 g, 4 mol) and methyl cyanoacetate (500 g, 5 mol) were dissolved in toluene (2500 mL). Then benzyl amine (40 g) and acetic acid (200 g) were added to the mixture in turn. The mixture was stirred at reflux for 15 h and the water formed was removed by azeotrope until no more water was formed. The mixture was cooled down, the solid formed in the mixture was filtered off and the cake was washed with toluene (100 mL) and methanol (300 mL). 522 g of pure off-white product was obtained with a yield of 50%.

### Example 7

### Synthetic Routine 2 for Reaction of 3,4-Dimethoxyacetophenone with Methyl Cyanoacetate

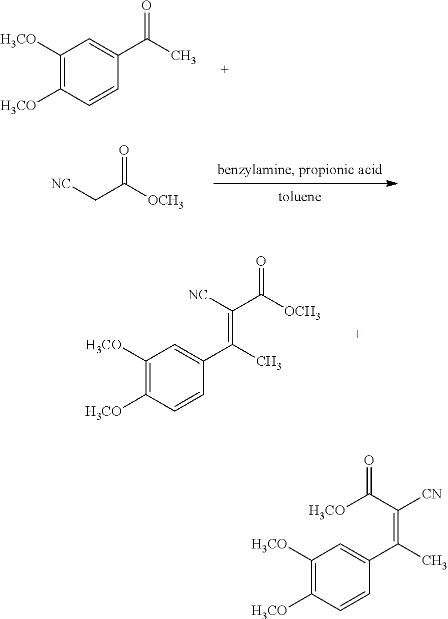 [SMILES: COc1ccc(C(C)=O)cc1OC ; COC(=O)CC#N ; COC(=O)/C(C#N)=C(\C)c1ccc(OC)c(OC)c1 ; COC(=O)/C(C#N)=C(/C)c1ccc(OC)c(OC)c1]

Batch 1: 3,4-dimethoxyacetophenone (90 g, 0.5 mol) and methyl cyanoacetate (60 g, 0.6 mol) were dissolved in toluene (400 mL). Then benzyl amine (6.6 g) and propionic acid (10 g) were added to the mixture in turn. The mixture was stirred at reflux for 10 h and the water formed was removed en vacuo through the formation of a binary azeotrope until no more water was formed. The mixture was cooled down, the solid precipitated in the mixture was filtered off (the filtrate could be applied for next batches while another part of benzyl amine and propionic acid is necessary) and the cake was rinsed with methanol (200 mL). 69 g of pure light yellow product was obtained with a yield of 59.5% (mp=170-174° C.).

Batch 2: The filtrate of the first batch was applied for this batch, and 3,4-dimethoxyacetophenone (90 g, 0.5 mol), methyl cyanoacetate (60 g, 0.6 mol), benzyl amine (6.6 g) and propionic acid (10 g) were added. 78 g of pure light yellow product was obtained and the yield was 67.2%.

Batch 3: The filtrate of the second batch was applied for this batch, and 3,4-dimethoxyacetophenone (90 g, 0.5 mol), methyl cyanoacetate (60 g, 0.6 mol), benzyl amine (6.6 g) and propionic acid (10 g) were added. 70 g of pure light yellow product was obtained and the yield was 60%.

Batch 4: The filtrate of the third batch was applied for this batch, only benzyl amine (6.6 g) and propionic acid (10 g) were added. 87 g of pure light yellow product was obtained.

3,4-dimethoxyacetophenone (270 g, 1.5 mol), methyl cyanoacetate (180 g, 1.8 mol) were consumed in the 4 batches, and 304 g of the final product was obtained and the total yield was 87.6%.

What is claimed is:

1. A photostabilized photoactive composition comprising:
at least one photoactive compound that develops an excited state energy when subjected to UV radiation; and
at least one para-alkoxyl phenyl substituted propenoic acid derivative of Formula I: 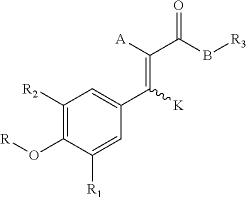
wherein:
R is selected from the group consisting of C₁-C₃₀ alkyl;
R₁ is selected from the group consisting of C₁-C₁₅ alkoxyl, OH, and H;
R₂ is selected from the group consisting of H and C₁-C₁₅ alkoxyl;
R₃ is selected from the group consisting of C₁-C₄₀ alkyl;
K is selected from the group consisting of C₁-C₁₅ alkyl;
B is selected from the group consisting of O or S; and
A is selected from the group consisting of CN or (C═O)NR₄(R₅),
wherein R₄ and R₅ are independently selected from C₁-C₁₅ alkyl;
wherein R, R₁, R₂, R₃, R₄, and R₅ may be either straight chain or branched chain;
wherein the at least one para-alkoxy phenyl substituted propenoic acid derivative of Formula I comprises the Z stereoisomer, the E stereoisomer, or a combination thereof; and
wherein the at least one para-alkoxyl phenyl substituted propenoic acid derivative of Formula I is operable to quench the excited state energy.

2. A photostabilized photoactive composition according to claim 1, wherein the at least one para-alkoxyl phenyl substituted propenoic acid derivative of Formula I is selected from the group consisting of: 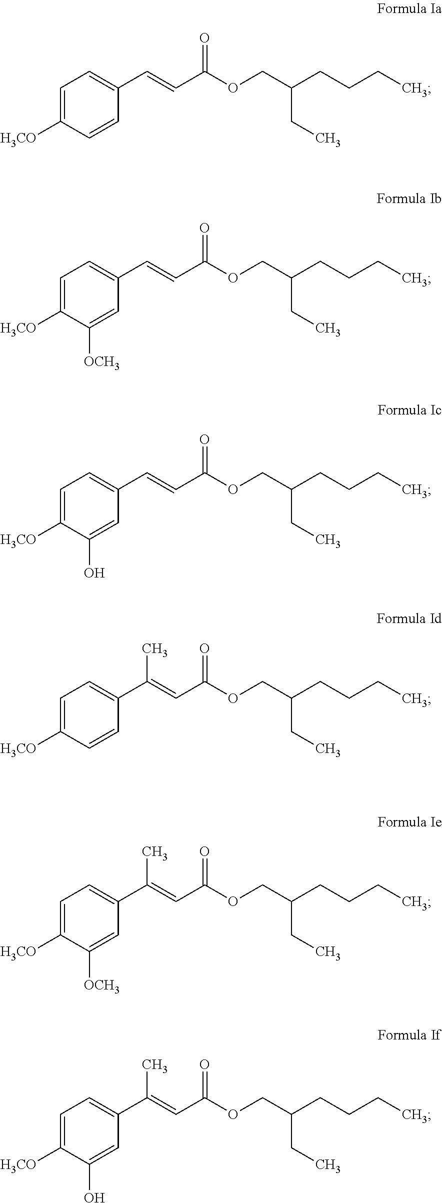 [SMILES: CCCCC(CC)COC(=O)/C=C/c1ccc(OC)cc1 ; CCCCC(CC)COC(=O)/C=C/c1ccc(OC)c(OC)c1 ; CCCCC(CC)COC(=O)/C=C/c1ccc(OC)c(O)c1 ; CCCCC(CC)COC(=O)/C=C(\C)c1ccc(OC)cc1 ; CCCCC(CC)COC(=O)/C=C(\C)c1ccc(OC)c(OC)c1 ; CCCCC(CC)COC(=O)/C=C(\C)c1ccc(OC)c(O)c1]
or
any combination thereof.

3. A photostabilized photoactive composition according to claim 1, wherein the at least one para-alkoxyl phenyl substituted propenoic acid derivative of Formula I is present at a concentration of about 0.000001% to about 20%, based on the total weight of the composition.

4. A photostabilized photoactive composition according to claim 1, wherein the at least one para-alkoxyl phenyl substituted propenoic acid derivative of Formula I is present at a concentration comprising about 15% by weight, about 10% by weight, about 5% by weight, about 1% by weight, about 0.5% by weight, about 0.1% by weight, or a combination thereof, based on the total weight of the composition.

5. A photostabilized photoactive composition of claim 2, wherein the at least one para-alkoxyl phenyl substituted propenoic acid derivative of Formula Ia, Ib, Ic, Id, Ie, If, or a combination thereof, is present at a concentration of about 0.000001% to about 20%, based on the total weight of the composition.

6. A photostabilized photoactive composition of claim 2, wherein the at least one para-alkoxyl phenyl substituted propenoic acid derivative of Formula Ia, Ib, Ic, Id, Ie, If, or a combination thereof, is present at a concentration comprising about 15% by weight, about 10% by weight, about 5% by weight, about 1% by weight, about 0.5% by weight, about 0.1% by weight, or a combination thereof, based on the total weight of the composition.

7. A photostabilized photoactive composition according to claim 1, wherein the at least one photoactive compound comprises 4-methyldibenzoylmethane and derivatives thereof; octyl methoxycinnamate and derivatives thereof; octocrylene and derivatives thereof; p-methoxycinnamic acid esters and derivatives thereof; 1-(4-methoxyphenyl)-3-(4-tert-butylphenyl)propane-1,3-dione and derivatives thereof; oxybenzone and derivatives thereof; bis-ethylhexyloxyphenol methoxyphenyl triazone and derivatives thereof; methylene bis-benzotriazolyl tetramethylbutylphenol and derivatives thereof; 4-methylbenzylidene camphor and derivatives thereof; diethylamino hydroxyl benzoyl hexyl benzoate and derivatives thereof; drometrizole trisiloxane and derivatives thereof; ethylhexyl triazone, diethylhexyl butamido triazone and derivatives thereof; terephthalylidene dicamphor sulfonic acid, its salts, and derivatives thereof, menthyl anthranilate and derivatives thereof; retinol and derivatives thereof; coenzyme Q and derivatives thereof; cholecalciferol and derivatives thereof; porphyrin and derivatives thereof, resveratrol and derivatives thereof; p-aminobenzoic acid, its salts, and derivatives thereof; glyceryl esters; anthranilate and derivatives thereof; cinnamic acid and derivatives thereof, coumarin and derivatives thereof; trihydroxycinnamic acid and derivatives thereof; dibenzalacetone naphtholsulfonate, its salts, and derivatives thereof; dibenzalacetone and derivatives thereof; dihydroxy-naphthoic acid, its salts, and derivatives thereof; o-hydroxydiphenyldisulfonate, its salts, and derivatives thereof; diazole derivatives; quinine derivatives, its salts, and derivatives thereof; quinoline derivatives; hydroxyl-substituted benzophenone derivatives; quinolone derivatives; benzophenone derivatives; uric acid derivatives; quinine salts; hydroxydiphenyldisulfonate, its salts, and derivatives thereof; tannic acid and derivatives thereof; violuric acid and derivatives thereof; phenylbenzimidazole tetrasulfonate, its salts, and derivatives thereof; terephthalylidene dicamphor sulfonic acid, its salts, and derivatives thereof; benzalacetophenone naphtholsulfonate, its salts, and derivatives thereof; benzoic acid, its salts, and derivatives thereof; o-hydroxydiphenyldisulfonate, its salts, and derivatives thereof; p-naphthalate derivatives; methoxy-substituted uric acid derivatives; hydroquinone, its salts, and derivatives thereof; benzophenone derivatives; dihydroxycinnamic acid, its salts, and derivatives thereof; 1, 3, 5-triazine derivatives; methylene bis-benzotriazolyl tetramethylbutylphenol, its salts, and derivatives thereof; titanium dioxide and derivatives thereof; triazole and derivatives thereof; zinc oxide; bis-ethylhexyloxyphenol methoxyphenyl triazine and its salts; salicylate and derivatives thereof; diethylamino hydroxyl benzoyl hexyl benzoate, its salts, and derivatives thereof; and combinations of the foregoing.

8. A photostabilized photoactive composition according to claim 1, wherein the at least one photoactive compound is present at a concentration of about 0.000001% to about 20%, based on the total weight of the composition.

9. A photostabilized photoactive composition according to claim 1, wherein the at least one photoactive compound is present at a concentration comprising about 15% by weight, about 10% by weight, about 5% by weight, about 1% by weight, about 0.5% by weight, about 0.1% by weight, or a combination thereof, based on the total weight of the composition.

10. A photostabilized photoactive composition according to claim 1, wherein said composition includes an oil phase having a dielectric constant of at least about 8.

11. A photostabilized photoactive composition according to claim 1, wherein said composition may enhance protection of at least one polymer against UV radiation, the at least one polymer comprising polyvinyl chloride, polystyrene, low-density polyethylene, high-density polyethylene, polyamides, nylon, polypropylene, rubber, and cellulose.

12. A photostabilized photoactive composition according to claim 1, wherein said composition may enhance protection of at least one coating against UV radiation, the at least one coating comprising adhesives, acrylic paint, latex paint, stains, caulk, sealants, urethanes, enamels, films, and inks.

13. A photostabilized photoactive composition according to claim 1, wherein said composition may enhance protection of a sunscreen against UV radiation, wherein application of said sunscreen to a skin of an animal (e.g., human) may protect the skin against UV radiation.

14. A photostabilized photoactive composition according to claim 1, wherein said composition may enhance protection of a cosmetic against UV radiation, wherein application of said cosmetic to a skin or a hair of an animal (e.g., human) may protect the skin or the hair against UV radiation.

15. A photostabilized photoactive composition according to claim 12, wherein application of said coating to a surface (e.g., polymer, metal, canvas, or wood) may protect the surface against UV radiation.

16. A method of prolonging UV absorption capabilities of a composition containing at least one photoactive compound and at least one para-alkoxyl phenyl substituted propenoic acid derivative, the method comprising:
adding the at least one para-alkoxyl phenyl substituted propenoic acid derivative of Formula I in an effective amount to the composition,
wherein the at least one para-alkoxyl phenyl substituted propenoic acid derivative of Formula I comprises: 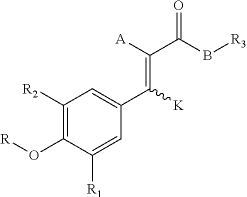
wherein:
R is selected from the group consisting of C₁-C₃₀ alkyl;
R₁ is selected from the group consisting of C₁-C₁₅ alkoxyl, OH, and H;
R₂ is selected from the group consisting of H and C₁-C₁₅ alkoxyl;
R₃ is selected from the group consisting of C₁-C₄₀ alkyl;
K is selected from the group consisting of C₁-C₁₅ alkyl;
B is selected from the group consisting of O or S; and
A is selected from the group consisting of CN or (C═O)NR₄(R₅),
wherein R₄ and R₅ are independently selected from C₁-C₁₅ alkyl;
wherein R, R₁, R₂, R₃, R₄, and R₅ may be either straight chain or branched chain;
wherein the at least one photoactive compound absorbs UV radiation and thereby protects a substrate from UV radiation;
wherein the at least one para-alkoxy phenyl substituted propenoic acid derivative of Formula I comprises the Z stereoisomer, the E stereoisomer, or a combination thereof; and
wherein the at least one para-alkoxyl phenyl substituted propenoic acid of Formula I is operable as an excited state energy acceptor.

17. A method according to claim 16, wherein the at least one para-alkoxyl phenyl substituted propenoic acid derivative of Formula I is selected from the group consisting of: 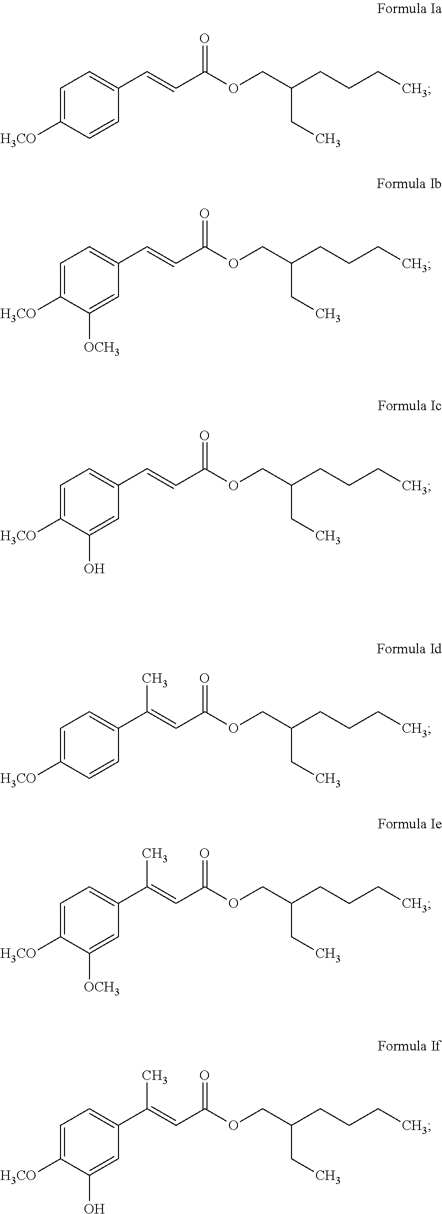 [SMILES: CCCCC(CC)COC(=O)/C=C/c1ccc(OC)cc1 ; CCCCC(CC)COC(=O)/C=C/c1ccc(OC)c(OC)c1 ; CCCCC(CC)COC(=O)/C=C/c1ccc(OC)c(O)c1 ; CCCCC(CC)COC(=O)/C=C(\C)c1ccc(OC)cc1 ; CCCCC(CC)COC(=O)/C=C(\C)c1ccc(OC)c(OC)c1 ; CCCCC(CC)COC(=O)/C=C(\C)c1ccc(OC)c(O)c1]
or
any combination thereof.

18. A method according to claim 16, wherein the substrate comprises a polymer, a coating, and a sunscreen.

19. A method according to claim 18, wherein the polymer comprises polyvinyl chloride, polystyrene, low-density polyethylene, high-density polyethylene, polyamides, nylon, polypropylene, rubber, and cellulose.

20. A method according to claim 18, wherein application of said sunscreen to a skin of an animal (e.g., human) thereby protects the skin against UV radiation.

21. A method according to claim 18, wherein the coating comprises adhesives, acrylic paint, latex paint, stains, caulk, sealants, urethanes, enamels, films, and inks.

22. A method according to claim 16, wherein the at least one para-alkoxyl phenyl substituted propenoic acid derivative of Formula I is present at a concentration of about 0.000001% to about 20%, based on the total weight of the composition.

23. A method according to claim 16, wherein the at least one para-alkoxyl phenyl substituted propenoic acid derivative of Formula I is present at a concentration comprising about 15% by weight, about 10% by weight, about 5% by weight, about 1% by weight, about 0.5% by weight, about 0.1% by weight, or a combination thereof, based on the total weight of the composition.

24. A method according to claim 16, wherein the at least one para-alkoxyl phenyl substituted propenoic acid derivative of Formula Ia, Ib, Ic, Id, Ie, If, or a combination thereof, is present at a concentration of about 0.000001% to about 20%, based on the total weight of the composition.

25. A method according to claim 16, wherein the at least one para-alkoxyl phenyl substituted propenoic acid derivative of Formula Ia, Ib, Ic, Id, Ie, If, or a combination thereof, is present at a concentration comprising about 15% by weight, about 10% by weight, about 5% by weight, about 1% by weight, about 0.5% by weight, about 0.1% by weight, or a combination thereof, based on the total weight of the composition.

26. A method according to claim 16, wherein the at least one photoactive compound comprises 4-methyldibenzoylmethane and derivatives thereof; octyl methoxycinnamate and derivatives thereof; octocrylene and derivatives thereof; p-methoxycinnamic acid esters and derivatives thereof; 1-(4-methoxyphenyl)-3-(4-tert-butylphenyl)propane-1,3-dione and derivatives thereof; oxybenzone and derivatives thereof; bis-ethylhexyloxyphenol methoxyphenyl triazone and derivatives thereof; methylene bis-benzotriazolyl tetramethylbutylphenol and derivatives thereof; 4-methylbenzylidene camphor and derivatives thereof; diethylamino hydroxyl benzoyl hexyl benzoate and derivatives thereof; drometrizole trisiloxane and derivatives thereof; ethylhexyl triazone, diethylhexyl butamido triazone and derivatives thereof; terephthalylidene dicamphor sulfonic acid, its salts, and derivatives thereof, menthyl anthranilate and derivatives thereof; retinol and derivatives thereof; coenzyme Q and derivatives thereof; cholecalciferol and derivatives thereof; porphyrin and derivatives thereof, resveratrol and derivatives thereof; p-aminobenzoic acid, its salts, and derivatives thereof; glyceryl esters; anthranilate and derivatives thereof; cinnamic acid and derivatives thereof, coumarin and derivatives thereof; trihydroxycinnamic acid and derivatives thereof; dibenzalacetone naphtholsulfonate, its salts, and derivatives thereof; dibenzalacetone and derivatives thereof; dihydroxy-naphthoic acid, its salts, and derivatives thereof; o-hydroxydiphenyldisulfonate, its salts, and derivatives thereof; diazole derivatives; quinine derivatives, its salts, and derivatives thereof; quinoline derivatives; hydroxyl-substituted benzophenone derivatives; quinolone derivatives; benzophenone derivatives; uric acid derivatives; quinine salts; hydroxydiphenyldisulfonate, its salts, and derivatives thereof; tannic acid and derivatives thereof; violuric acid and derivatives thereof; phenylbenzimidazole tetrasulfonate, its salts, and derivatives thereof; terephthalylidene dicamphor sulfonic acid, its salts, and derivatives thereof; benzalacetophenone naphtholsulfonate, its salts, and derivatives thereof; benzoic acid, its salts, and derivatives thereof; o-hydroxydiphenyldisulfonate, its salts, and derivatives thereof; p-naphthalate derivatives; methoxy-substituted uric acid derivatives; hydroquinone, its salts, and derivatives thereof; benzophenone derivatives; dihydroxycinnamic acid, its salts, and derivatives thereof; 1, 3, 5-triazine derivatives; methylene bis-benzotriazolyl tetramethylbutylphenol, its salts, and derivatives thereof; titanium dioxide and derivatives thereof; triazole and derivatives thereof; zinc oxide; bis-ethylhexyloxyphenol methoxyphenyl triazine and its salts; salicylate and derivatives thereof; diethylamino hydroxyl benzoyl hexyl benzoate, its salts, and derivatives thereof; and combinations of the foregoing.

27. A method according to claim 16, wherein the at least one photoactive compound is present at a concentration of about 0.000001% to about 20%, based on the total weight of the composition.

28. A method according to claim 16, wherein the at least one photoactive compound is present at a concentration comprising about 15% by weight, about 10% by weight, about 5% by weight, about 1% by weight, about 0.5% by weight, about 0.1% by weight, or a combination thereof, based on the total weight of the composition.

29. A method according to claim 16, wherein said composition includes an oil phase having a dielectric constant of at least about 7.

30. A method according to claim 16, wherein said composition includes an oil phase having a dielectric constant of at least about 8.

31. A method according to claim 16, wherein application of said cosmetic to a skin or a hair of an animal (e.g., human) thereby protects the skin or the hair against UV radiation.

32. A method according to claim 16, wherein application of said coating to a surface (e.g., polymer, metal, canvas, or wood) thereby protects the surface against UV radiation.
